(19) United States
(12) Patent Application Publication
(10) Pub. No.: US 2026/0122324 A1
(43) Pub. Date: Apr. 30, 2026

(54) METHOD AND SYSTEM FOR GENERATING CONTENT FOR GUIDED REFLECTION

(71) Applicant: Sequelae, Inc., Minneapolis, MN (US)

(72) Inventors: Rathish Mohan, Bothell, WA (US); Aniruddha Chandrashekhar Gaikwad, Cary, NC (US); Danielle Marria Martin, Minneapolis, MN (US); Natalie Sheils, St. Paul, MN (US); Jonathon David Gaff, Auburn, WA (US); Benjamin Jacob Bischoff, Provo, UT (US); Jason Lamont Carter, Chapel Hill, NC (US); Sara Taylor, Edina, MN (US); Steven J. Catani, Athens, GA (US)

(73) Assignee: Sequelae, Inc., Minneapolis, MN (US)

(21) Appl. No.: 19/368,301

(22) Filed: Oct. 24, 2025

Related U.S. Application Data
(60) Provisional application No. 63/712,176, filed on Oct. 25, 2024.

(51) Int. Cl. H04N 21/854; G06F 16/334

(52) U.S. Cl. CPC H04N 21/854; G06F 16/334

(57) ABSTRACT

Systems and methods for generating content for guided reflection are provided. A digital communication network (DCN) is for a plurality of members of a population to communicate with at least each other or the DCN. An individual member is identified with an adverse health condition. A request is transmitted to the individual member to select a topic of reflection from a plurality of topics of reflection corresponding to the adverse health condition. Content is automatically generated comprising at least one of audible content, visual content, or an audible and visual content based on the selected topic of reflection. The content is designed to cause the individual member to connect an outcome experienced by the individual member with a decision the individual member has made. A communication is generated and transmitted to the individual member that includes the content and the selected topic of reflection.

### CROSS REFERENCE TO RELATED APPLICATIONS

This application claims the benefit of U.S. Provisional Application No. 63/712,176, filed on Oct. 25, 2024, which application is incorporated herein by reference in its entirety.

### BACKGROUND

Various embodiments relate generally to methods and systems for generating content and in particular, for generating content for guided reflection.

This section is intended to provide a background or context. The description may include concepts that may be pursued, but have not necessarily been previously conceived or pursued. Unless indicated otherwise, what is described in this section is not deemed prior art to the description and claims and is not admitted to be prior art by inclusion in this section.

People often take actions—whether simple, hard, good, or bad, etc.—that impact their health. Regardless, such actions have consequences that can accumulate over time. However, rarely do people associate such actions with consequences. This may be due to, for example, the lack of data to see the consequence (or the data is available in a way that does not connect to the action). Other times, they may not think about the connection between the action and the consequence. Reflecting on their conditions can help people associate actions with their consequences; thus, it is desirable for people to reflect on their actions and potential consequences.

Mindfulness, as gained through reflection, is an essential part of self-improvement. Unless someone is aware of their behaviors and how those behaviors impact the things that are important to them, it is very hard to sustain change in their behaviors.

Reflection is the process by which people gain that understanding. Reflecting takes conscience effort and is often emotionally draining and cognitively draining. Thus, a person may find it easier to not reflect on such actions.

Reflection can be based on more than text or verbal conversations. People can use artistic expressions including, for example, non-prose material such as poetry, music, or art to put them into a mindset to consider and reflect their situation. However, as each person is distinct, it may be difficult to determine what materials might be best suited for each person.

Computers have changed the way people interact. Digital networks, which may include the use of social media, allow individuals to interact with others online and connect many people with a community. The online community can be utilized to help build positive behaviors and encourage people to make improvements in their lives. Computers can also provide a way for people to record their actions and review them later as well as to enable a person to reflect on their activities.

What is needed is a way to generate non-prose content for use in guided reflection via a digital communication network.

### SUMMARY

Example aspects of the present disclosure include:

A method according to at least one embodiment of the present disclosure comprises providing a digital communication network (DCN) for a plurality of members of a population to communicate with at least each other or the DCN; identifying, by a server controller, an individual member with an adverse health condition of the plurality of members; transmitting, by the server controller, a request to the individual member, via the DCN, to select a topic of reflection from a plurality of topics of reflection corresponding to the adverse health condition; automatically generating, by the server controller, content comprising at least one of audible content, visual content, or an audible and visual content based on the selected topic of reflection, the content designed to cause the individual member to connect an outcome experienced by the individual member with a decision the individual member has made; and transmitting a communication generated by the server controller to the individual member via the DCN, the communication including the content and the selected topic of reflection.

Any of the aspects herein, wherein the content is a first content and the communication generated by the server controller is a first communication, wherein the method further comprises: receiving a response from the individual member to the first communication generated by the server controller via the DCN; generating, by the server controller, a second content in response to receiving the response; and transmitting a second communication generated by the server controller to the individual member via the DCN, the second communication including the second content.

Any of the aspects herein,, wherein the second content is generated based on previous communications between the individual member and a conversation bot of the DCN.

Any of the aspects herein, further comprising: generating, by the server controller, a question designed to cause the individual member to associate an action with an outcome experienced, wherein the communication generated by the server controller includes the question.

Any of the aspects herein, further comprising: analyzing, by the server controller, one or more communications from the individual member, wherein the content is automatically generated based in part of the analysis of the one or more communications.

Any of the aspects herein, further comprising: generating, by the server controller, a prompt containing at least the selected topic of reflection and instructions to generate the topic, wherein automatically generating the content comprises prompting a large language model with the prompt and receiving the content as output from the large language model.

Any of the aspects herein, wherein the plurality of topics of reflection comprises a biometric reading, a psychological or emotional state, an inspirational quotation identified as useful to the individual member, music, movies, art, prose, or poetry identified as appreciated by the individual member, a daily routine, a life event, a time of year, a season, a time of day, a diagnosis, an injury, an elective medical procedure, a current challenge which the individual member is experiencing, a current challenge which the individual member has taken on, a relationship, a planned trip, and an unplanned trip.

Any of the aspects herein, further comprising: analyzing, by the server controller, one or more communications from the individual member; and generating, by the server controller, the plurality of topics of reflection based on the analysis of the one or more communications.

Any of the aspects herein, further comprising: analyzing, by the server controller, one or more communications from the individual member; and determining, by the server controller, the adverse health condition based on the analysis of the one or more communications.

Any of the aspects herein, wherein the content comprises at least one of a piece of music, a piece of art, a lyrical piece of music, a line of a song, a line of an opera, a video showing a choreographed dance, a song, a painting, a mural, mixed media content, and a combination of music with a piece of art.

A system according to at least one embodiment of the present disclosure comprises a computer processor; a data repository in communication with the computer processor and storing: a request; a plurality of topics of reflection; a content; and a communication; a digital communications network (DCN) which, when executed by the computer processor, provides a network for a plurality of members of a population to interact with at least each other or the DCN; and a server controller which, when executed by the computer processor: identifies an individual member with an adverse health condition from the plurality of members; transmits the request to the individual member, via the DCN, to select a topic of reflection from the plurality of topics of reflection corresponding to the adverse health condition; automatically generates content comprising at least one of audible content, visual content, or an audible and visual content based on the selected topic of reflection, the content designed to cause the individual member to connect an outcome experienced by the individual member with a decision the individual member has made; and transmits the communication to the individual member via the DCN, the communication including the content and the selected topic of reflection.

Any of the aspects herein, wherein the content is a first content and the communication is a first communication, and wherein the server controller when further executed by the processor: receives a response from the individual member to the first communication via the DCN; generates a second content in response to receiving the response; and transmits a second communication to the individual member via the DCN, the second communication including the second content.

Any of the aspects herein, wherein the second content is generated based on previous communications between the individual member and a conversation bot of the DCN.

Any of the aspects herein, wherein the server controller when further executed by the processor: generates a question designed to cause the individual member to associate an action with an outcome experienced, wherein the communication generated by the server controller includes the question.

Any of the aspects herein, wherein the server controller when further executed by the processor: analyzes one or more communications from the individual member, wherein the content is automatically generated based in part of the analysis of the one or more communications.

Any of the aspects herein, wherein the server controller when further executed by the processor: generates a prompt containing at least the selected topic of reflection and instructions to generate the topic, wherein automatically generating the content comprises prompting a large language model with the prompt and receiving the content as output from the large language model.

Any of the aspects herein, wherein the plurality of topics of reflection comprises a biometric reading, a psychological or emotional state, an inspirational quotation identified as useful to the individual member, music, movies, art, prose, or poetry identified as appreciated by the individual member, a daily routine, a life event, a time of year, a season, a time of day, a diagnosis, an injury, an elective medical procedure, a current challenge which the individual member is experiencing, a current challenge which the individual member has taken on, a relationship, a planned trip, and an unplanned trip.

Any of the aspects herein, wherein the server controller when further executed by the processor: analyzes one or more communications from the individual member; and generates the plurality of topics of reflection based on the analysis of the one or more communications.

Any of the aspects herein, wherein the server controller when further executed by the processor: analyzes one or more communications from the individual member; and determines the adverse health condition based on the analysis of the one or more communications.

Any of the aspects herein, wherein the content comprises at least one of a piece of music, a piece of art, a lyrical piece of music, a line of a song, a line of an opera, a video showing a choreographed dance, a song, a painting, a mural, mixed media content, and a combination of music with a piece of art.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE DRAWINGS

Aspects of the described embodiments are more evident in the following description, when read in conjunction with the attached Figures.

FIG. 1 shows a simplified block diagram of devices, in accordance with one or more embodiments.

FIG. 2 is a logic flow diagram that illustrates the operation of a method, in accordance with one or more embodiments.

FIG. 3A shows an example of a computing system, in accordance with one or more embodiments.

FIG. 3B shows an example of a network, in accordance with one or more embodiments.

### DETAILED DESCRIPTION

Reflection is normally considered after an event. A person can observe a change and reflect on the cause of that change. This reflection helps understand why a change, good or bad, had occurred and can teach the person how to achieve better results. Often these changes are observed through objective data, such as, a biometric sample, results tracked through a smartwatch, or some other means that bring the change to life.

Reflections are also an important part of a person's experience. They allow the person to turn observations about the connection between their actions and the consequences of those actions into useable insight. Digital health applications typically provide people access to data about their health or health-related actions and often allow them to record observations about how they feel. This can cause reflection but in a minimal way.

Reflection may be considered as part of the change process—a person does or observes something, and then reflects on the action or observation and the consequences. This process implies some degree of planning, either in adding reflection to a planned action, or scheduling periods to reflect (these can be formal as in professional counseling). Unfortunately, much of life is unplanned and unobserved.

A person can reflect on many aspects of their experience. The topic of their reflection can help the person focus on specific elements. These elements can improve the reflection process, for example, by enabling the person to make a personal connection with the reflections, such as, associating the process with an inspirational quote, music, movie, etc.

The topic may also highlight parts of the person's life that may be impacted by the lifestyle condition. The topic may help the person identify changes in their daily routines, the time of day, the time of year and/or upcoming life events due to their lifestyle condition. Conversely, the person may reflect upon how the lifestyle condition may be influenced by their daily routines, the time of day, the time of year and/or upcoming life events.

Various embodiments overcome those problems, for example, by determining or generating content such as non-prose content for an individual member and using such content to encourage the individual member to reflect on a topic of reflection. By guiding the reflection, the person reflecting on their lifestyle condition can better understand their situation and can be better positioned to handle their lifestyle condition.

Further, in some embodiments, the DCN presents an individual member with opportunities to reflect on a topic. The DCN may send a message to the individual member requesting a response regarding the topic. The message may be part of a conversion the individual member is having with a bot in the DCN. Additionally, the DCN may incentivize the reflection, for example, offering rewards for responding. The rewards may be an offer to participate in events, discounts, messaging icons, etc.

The DCN can analyze the individual member's communications in the DCN in order to select a topic for reflection. This can help identify aspects that the individual member is passionate about and/or shows specific interest in. The DCN may also base the topic on various other factors, for example the individual member's phenotype, the lifestyle condition, biological test results, family history, etc.

The DCN may also time the reflection requests so as to improve the reflection. As one non-limiting example, the DCN may analyze posts about what the individual member is eating to determine their eating cycle. The DCN can then time sending a request for the member to reflect on their lifestyle condition at a point shortly after the individual member is expected to have completed a meal.

The DCN may also follow-up on the communications by requesting additional reflection from the individual member. For example, the DCN may ask the individual member to reflect on the factors that caused their response to the automated message. The individual member may respond to the follow-up request which may lead to further communications. These communications may occur over a single session or multiple sessions. Multiple sessions may take place over a single day or several days (or weeks).

The individual member's responses may be provided in various ways, for example, textual, verbal, visual, or some combination thereof. The DCN may provide a tool for the member to provide the responses, such as a voice capture program to translate speech into text. The individual member may provide detailed impressions, indicate their expected results and/or indicate a level of comfort (or expected level of comfort) with the results.

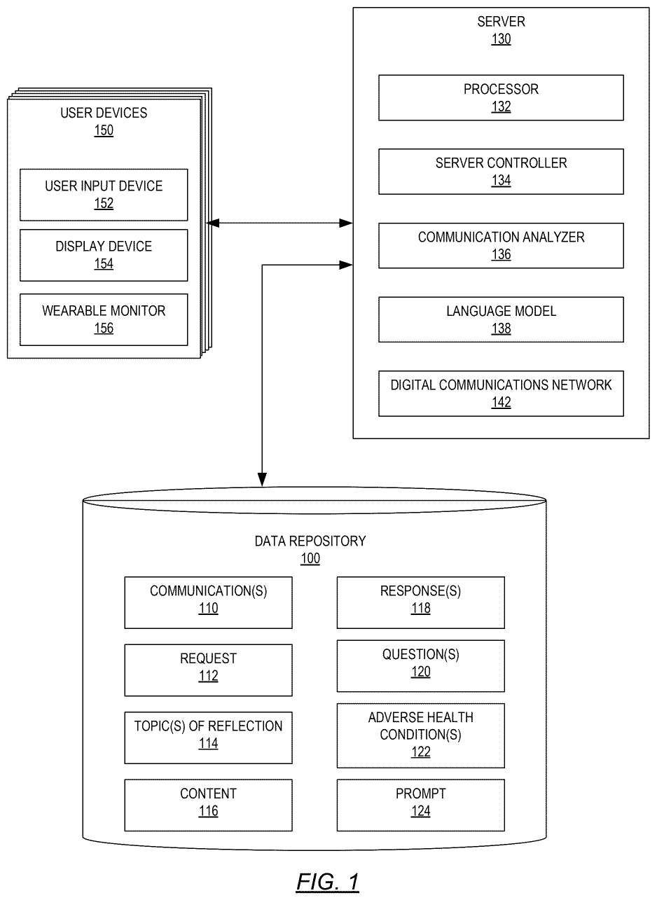

The system shown in FIG. 1 includes a data repository (100). The data repository (100) is a type of storage unit or device (e.g., a file system, database, data structure, or any other storage mechanism) for storing data (described below). The data repository (100) may include multiple different, potentially heterogeneous, storage units and/or devices.

The data repository (100) stores communication(s) (110). The communications (110) are communications from members in a digital communication network (DCN) (142) (described in more detail below) or communications (110) transmitted or sent via the DCN (142) to member(s) of the DCN (142). The communications (110) may be, for example, text-based, image-based, multimedia communications, audio communications, or any combinations thereof. The communications may be between members, communications in a forum, communications with the DCN (142), or communications generated by, for example, a conversation bot or a server controller (134) (discussed in more detail below) and transmitted by the DCN (142) (e.g., communications from a conversation bot, also referred to as a chat bot).

The communications (110) may also be referred to as bot communications when the communications are generated and received from one or more conversation bots of the DCN (142).

The data repository (100) also stores request(s) (112). The request (112) may be a communication (110) that includes a request (112) to an individual member to respond to the request (112). The request (112) may include asking the individual member to select a topic of reflection (114) or to select the topic of reflection (114) out of a set of topics of reflection (114). The request (112) may also include, in some embodiments, an incentive to encourage the individual member to respond to the request (112). The incentives can be, for example, discounts, coupons, money, or other rewards to incentivize the individual member to respond to the request (112). The incentives may be, for example, a discount or may cover the cost for an activity or an item.

The data repository (100) also stores topic(s) of reflection (114). The topics of reflections (114) can include, for example, an inspirational quote the individual member has identified as useful; a piece of music, a movie, a piece of art, a selection of prose, or a piece of poetry the individual member has identified as appreciating; a daily routine a life event; a time of year; a season; a time of day; a diagnosis; an injury; an elective medical procedure; a current challenge which the individual member has given themselves or is experiencing; a relationship; or a planned or unplanned trip. A particular topic of reflection (114) can be selected by the individual member. In other embodiments, the topic of reflection (114) can be determined by the DCN (142) analyzing communications (110) from the individual member. In such embodiments, the DCN (142) may use, for example, a communication analyzer (136) (described in more detail below) or the server controller (134) to analyze the communications (110). The communications (110) can be analyzed by, for example, identifying key words or concepts in the communications (110).

The topic of reflection (114) can also correspond to an adverse health condition (122) (discussed in detail below) of an individual member. For example, the topic of reflection (114) can include reflecting on whether the individual member's sleep routine positively or negatively affects their adverse health condition (122). In another example, the topic of reflection (114) can include reflecting on whether the individual member's diet positively or negatively affects their adverse health condition (122).

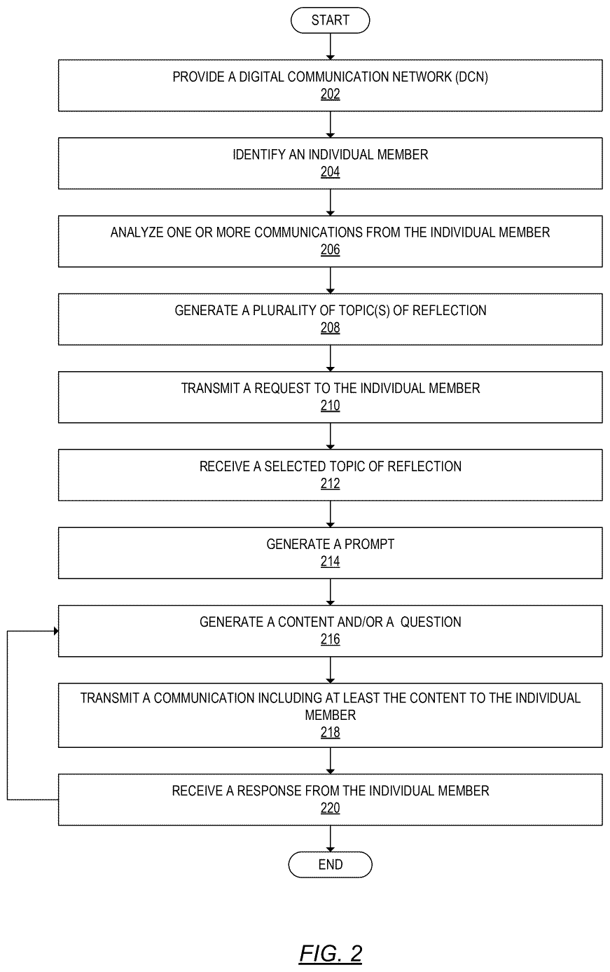

The data repository (100) also stores content (116). The content (116) can be audible content, visual content, or a combination of audible and visual content. For example, the content (116) can be a piece of music, a piece of art, a lyrical piece of music, a line of a song, a line of an opera, a video showing a choreographed dance, a song, a painting, a mural, mixed media content, and/or a combination of music with a piece of art. The content (116) can be generated automatically based on the selected topic of reflection (114) as will be discussed in more detail in FIG. 2.

The data repository (100) also stores response(s) (118). The responses (118) are responses (118) to the request (112) received from the individual member. The responses (118) may include, for example, a yes/no answer, text, and/or media (e.g., audio, visual, images, videos, etc.). The responses (118) can be used to generate additional or follow up requests (112), (content (116), and/or questions (120). The additional or follow up questions can be used to, for example, solicit additional responses (118).

The data repository (100) also stores question(s) (120). The questions (120) are questions directed to the individual member and may include yes/no questions or open-ended questions. The questions (120) may be designed to induce a reflection in the individual member, understand a motivation of the individual member, collect information about the life and lifestyle of the individual member, and/or cause the individual member to connect an outcome experienced by the individual member with a decision made by the individual member or an action the individual member has taken. The questions (120) can include one question, two questions, or more than two questions. The questions (120) may also include a request for the individual member to answer the question (120). The request may include buttons (e.g., yes/no buttons), a text and/or media box for the individual member to type in or attach media.

The data repository (100) also stores adverse health condition(s) (122) information. The adverse health condition (122) information stored can be information about the adverse health condition (122) that can be used to identify an adverse health condition in an individual member. For example, key words associated with the adverse health condition (122) can be stored. The key words can be used when, for example, analyzing communications (110) by the individual member to determine if the individual member has the associated adverse health condition (122). The adverse health condition (122) can be, for example, chronic systemic inflammation (CSI); malaise or low-energy; and/or a disease. The disease may be a prodromal disease, Obesity, Diabetes, rheumatoid arthritis, Crohn's, Psoriasis, eczema, cardiovascular disease, congestive heart failure, chronic obstructive pulmonary disease, asthma, depression, or anxiety. In other cases, the adverse health condition (122) may be a health risk such as falling or becoming infected with an illness or disease. In some cases, the adverse health condition (122) may be a social dysfunction or a prodromal disease.

The data repository (100) also stores prompts (124). The prompt (124) is a set of data that can be interpreted and understood by a language model (138) (described below) and that describes a desired output of the language model (138). The prompt (124) can include, for example, natural language text and/or media (i.e., images or video), as well as a command to describe the desired output. Additionally, the prompt (124) also may include example(s) of the desired output for the language model (138).

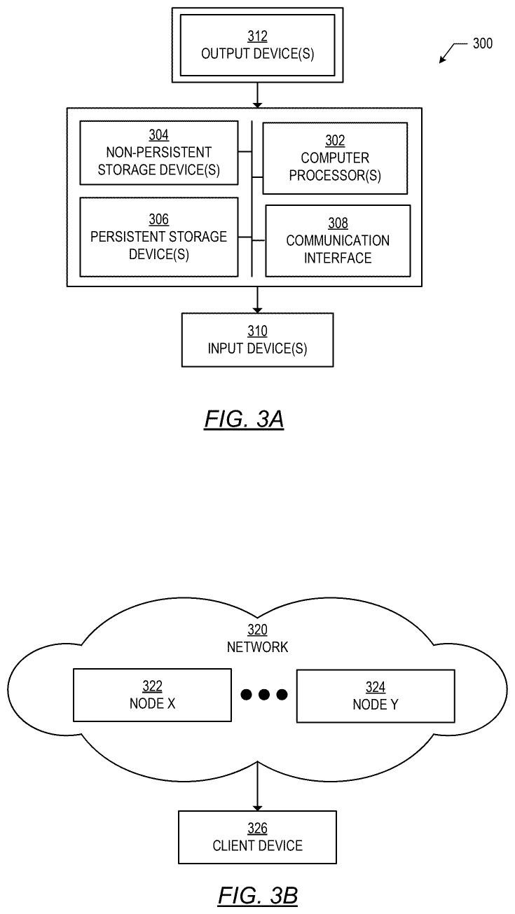

The system shown in FIG. 1 may include other components. For example, the system shown in FIG. 1 also may include a server (130). The server (130) is one or more computer processors, data repositories, communication devices, and supporting hardware and software. The server (130) may be in a distributed computing environment. The server (130) is configured to execute one or more applications, such as a communication analyzer (136) and/or the language model (138). An example of a computer system and network that may form the server (130) is described with respect to FIG. 3A and FIG. 3B.

The server (130) also includes a computer processor (132). The computer processor (132) is one or more hardware or virtual processors which may execute computer readable program code that defines one or more applications, such as the communication analyzer (136) and/or the language model (138). An example of the computer processor (132) is described with respect to the computer processor(s) (302) of FIG. 3A.

The server (130) also may include a server controller (134). The server controller (134) is software or application specific hardware which, when executed by the computer processor (136), controls and coordinates operation of the software or application specific hardware described herein. Thus, the server controller (134) may control and coordinate execution of the communication analyzer (136) and/or the language model (138).

The server (130) also includes the communication analyzer (136). The communication analyzer (136) is software or application specific hardware which, when executed by the computer processor (132), analyzes communication (110) in the DCN (142) and can generate questions (120), content (116), and/or the topics of reflection (114). The communication analyzer (136) can also be used to analyze the communications (110) from the individual member and identify the adverse health condition (122) of the individual member.

The server (130) may also include the language model (138). The language model (138) is a natural language processing machine learning model. An example of the language model (138) may be a large language model (LLM), such as CHATGPT®. However, many different language models may be used.

The server (130) also includes the DCN (142). The DCN (142) is a network through which members of a population can interact with each other, or with a system supported by the DCN (142). The DCN (142) can also interact with the members of the population and, for example, transmit communications (110) and the questions (114) to the individual member to induce reflection in the individual member.

In some embodiments, the DCN (142) may be, for example, an artificial intelligence bot configured to derive generated communications from analysis of existing communications (110) in the DCN (142) or biometrics provided to the DCN (142). Biometric data can be used in many instances such as the provisioning and commissioning of healthcare. For example, biometric data can be used to diagnose diabetes, prescribe hypertension medication with a patient's blood pressure exceeds a certain value, prescribe cancer drugs, etc. In another example, cancer drug can be available for reimbursement if a certain genetic signature is present in the tumor. The decision tree and other logic behind these relationships considers many things including what is known about the variance in the intervention's efficacy, therapeutic index, the accuracy and precision in the biometric data's measurement, as well as the cost-benefit ratio of the intervention compared with other interventional options.

The biometric data, as well as reflections from the individual member, can be used in a “feedback” decision tree where, for example, an intervention can be prescribed or recommended to an individual member based on the biometric data and/or the reflections. Then the biometric data or additional reflections can be obtained to study the effects or results of the intervention.

In some embodiments, the conversation bot may use a visual or graphical interface, for example, a rendered avatar, or a plot or image from the bot or sharing one with the bot. The DCN (142) can send a visual representation showing that person's data from a wearable, their interaction with the community, their consistency in talking with the bot, etc. The conversation bot may then ask questions like “What do you notice” or “Is anything here surprising”in order to induce a reflection from the individual member.

The conversation bot can analyze data from various sources, in part, to formulate its communications with the individual member. These sources include data from i) the DCN (142), ii) the ongoing conversation with the individual member, and iii) previous conversations with the individual member. The conversation bot can also use temporal and situational cues to formulate its communication next with the member. When analyzing the data, the conversation bot may quote sections of the conversations for use with communications between the individual member and/or the conversation bot may identify patterns in the discussions. When formulating the communications, the conversation bot can analyze exchanges in the DCN (142) from other members. These communications can be limited to specifically target individuals, such as those deemed to be close contacts to the individual member or members that are considered influencers.

One-to-one communications with the conversation bot can be triggered and informed by one-to-many communications in the DCN (142), and conversations with the conversation bot can (a) refer individuals to the relevant communications in the DCN (142), (b) suggest posts to the DCN (142), and (c) make reference to ideas contained in posts. The two ways to interact can behave in a synergistic way and allow the conversation bot to include a “human in the loop”in a surprisingly scalable way.

During the communication between the individual member and the conversation bot, the DCN (142) can analyze the individual member's responses. Based on the input from the individual member, the DCN (142) may identify an adverse health condition or a lifestyle condition for the individual member. The conversation bot may then gather more information regarding the adverse health condition, for example, to develop more details to confirm the adverse health condition. The communication may be based on related experiences from another member of the population. The conversation bot may quote content from a post by that other member.

The synergy between the conversation bot and the DCN (142) can facilitate reflection on a topic. Alternatively, this can be seen to be reversed where the act of facilitating reflection synergizes with usage of the bot in the DCN (142).

Using analysis of the individual member's communications in the DCN (142), the conversation bot can suggest a reflection on a topic in a one-to-one communication. The suggestion may be made as part of a question to the individual member. The conversation bot may then focus its conversations with that individual member on that topic for a period of time (e.g., a number of exchanges, a few minutes, a few days, etc.). The conversation bot can incorporate excerpts of communication on the DCN (142) in its responses. The excerpts of communications may include post titles, post topics, quotes from the body of the communication, and/or post contributors. Also, the excerpts could be summaries of communications on the DCN (142). These summaries may be created automatically by the DCN (142) and/or are based on a topic, a member's communications, or a time period.

The bot may prompt the individual member to reflect on various classes of action. The action may be active or passive. The action may also be one that was contemplated even if not undertaken.

Two or more conversation bots, each with a different personality or a different role (e.g. facilitate reflections, find useful communications, say good morning and inspire a positive start to a day, handle routine network tasks, provide custom communication summaries), may be provided by the DCN (142). The family of bots could each use conversations between the other bots and the user on the DCN (142) to formulate their responses. When working with an individual member, the bots may be selected based on the personality of the bot that is expected to work best with that user. The decision may be based on past dealings with the individual member and/or through analysis of that individual member's communication on the DCN (142). For example, when attempting to induce a reflection, the bot may use a first personality, then switch to a second personality when helping draft a post regarding that reflection. Accordingly, various embodiments can be used to provide a method to improve the communications of the conversation bot through using authentic experiences of the individual member in the communication.

The system shown in FIG. 1 also may include one or more user devices (150). The user devices (150) may be considered remote or local. A remote user device is a device operated by a third-party (e.g., an end user of a chatbot) that does not control or operate the system of FIG. 1. Similarly, the organization that controls the other elements of the system of FIG. 1 may not control or operate the remote user device. Thus, a remote user device may not be considered part of the system of FIG. 1.

In contrast, a local user device is a device operated under the control of the organization that controls the other components of the system of FIG. 1. Thus, a local user device may be considered part of the system of FIG. 1.

In any case, the user devices (150) are computing systems (e.g., the computing system (300) shown in FIG. 3A) that communicate with the server (130). The user devices (150) may include a wearable monitor (156). In an alternative embodiment, a separate wearable device may be in communication with the user device (150), such as a smart watch, or blood pressure monitor. The user devices (150) may also include a user input device (152) and/or a display device (154).

While FIG. 1 shows a configuration of components, other configurations may be used without departing from the scope of one or more embodiments. For example, various components may be combined to create a single component. As another example, the functionality performed by a single component may be performed by two or more components.

FIG. 2 is a logic flow diagram that illustrates a method, and a result of execution of computer program instructions, in accordance with various embodiments. The method can be used to guide reflection in an individual member using content automatically generated based on input from the individual member. The method can also, in other instances, be used in part to facilitate or accomplish at least one of: manage the health care cost of the population; reduce the health care risk in the population; and/or slow the progression of an adverse health condition. The adverse health condition can be, for example, chronic systemic inflammation, malaise, low energy, a disease, a health risk, social dysfunction, or a prodromal disease. In embodiments where the adverse health condition is a disease, the disease can be, for example obesity, diabetes, rheumatoid arthritis, Crohn's disease, psoriasis, eczema, cardiovascular disease, congestive heart failure, chronic obstructive pulmonary disease, asthma, depression, or anxiety. In embodiments where the adverse health condition is a health risk, the health risk can be, for example falling or becoming infected with an illness.

At Block 202, a step of providing a digital communication network (DCN) to a population is provided. The DCN may be the same as or similar to the DCN (142). As previously described, members of the population enrolled in the DCN can communicate with each other or with the DCN itself. The members of the population can also communication with one or more conversation bots.

At Block 204, a step of identifying an individual member is provided. The individual member may be identified based on communication(s) such as the communication(s) (110) from the individual member. The communication may be the same as or similar to the communication (110). In some embodiments, the communications may be a post by the individual member in the DCN and the post includes the at least one observation.

The individual member may be identified by, for example, the DCN or a server controller such as the server controller (134). The individual member may be identified based on, for example, their recent participation in an activity, posts by the individual member indicating that the individual member recently made a decision based on their health, etc.

At Block 206, a step of analyzing communications from the individual member is provided. The communications may be analyzed by, for example, a communication analyzer such as the communication analyzer (136).

At Block 208, a step of generating a plurality of topics of reflection is provided. In some embodiments, the communication analyzer may identify or determine the topics of reflection, which may be the same as or similar to the topics of reflection (114) based on the communications and/or the observation. The topics of reflection may be determined by, for example, identifying key words or concepts in the communications. As previously described, the topics of reflection may include, for example, an inspirational quote the individual member has identified as useful; a piece of music, a movie, a piece of art, a selection of prose, or a piece of poetry the individual member has identified as appreciating; a daily routine a life event; a time of year; a season; a time of day; a diagnosis; an injury; an elective medical procedure; a current challenge which the individual member has given themselves or is experiencing; a relationship; or a planned or unplanned trip.

In some embodiments, the method may not include the Block 208.

At Block 210, a step of transmitting a request to the individual member is provided. The request may be the same as or similar to the request (112). The request may include instructions or a request to the individual member to select a topic of reflection from the plurality of topics of reflections corresponding to an adverse health condition of the individual member. The adverse health condition may be the same as or similar to the adverse health condition (122).

In some embodiments, the method may include identifying the adverse health condition of the individual member based on the analysis of communications conducted at Block 206. For example, key words associated with the adverse health condition can be used to determine or identify if the individual member has the associated adverse health condition.

At Block 212, a step of receiving a selected topic of reflection is provided. The topic of reflection may be received from the individual member via the DCN.

At Block 214, a step of generating a prompt is provided. The prompt may be the same as or similar to the prompt (124). The prompt can include, for example, natural language text and/or media (i.e., images or video), as well as a command to describe the desired output. Additionally, the prompt also may include example(s) of the desired output for a language model such as the language model (138). The prompt can also includes at least the selected topic of reflection and instructions to generate content such as the content (116).

In some embodiments, the method may not include the Block 214.

At Block 216, a step of generating content and/or a question is provided The question may be the same as or similar to the question (120). In some embodiments, automatically generating the content and/or the question includes prompting the language model with the prompt and receiving the content and/or the questions as output from the language model.

The content may include an audible content, visual content, or an audible and visual content based on the selected topic of reflection. For example, the content may include a piece of music, a piece of art, a lyrical piece of music, a line of a song, a line of an opera, a video showing a choreographed dance, a song, a painting, a mural, mixed media content, and/or a combination of music with a piece of art.

The content and/or the question may each be designed to cause the individual member to connect an outcome experienced by the individual member with a decision the individual member has made and related to their adverse health condition. For example, the adverse health condition may be fatigue, the content may be a song about taking action, and the question may ask the individual member whether improving their sleep habits has helped with their fatigue.

At Block 218, a step of transmitting a communication including at least the content to the individual member is provided. The communication may be generated by, for example, the server controller and transmitted to the individual member by the DCN. The communication includes the content and/or the questions. The communication may also include, in some embodiments, instructions and/or an incentive for the individual member to respond to the communication.

At Block 220, a step of receiving a response from the individual member is provided. The response may be received via the DCN and may include, for example, answers to the questions and/or reflections on the questions and/or content. In some embodiments, the response may be posted to the DCN by the DCN. The response may be posted to encourage other members of the population to reflect on the connection between their own outcome(s) and decisions or actions taken by the other members. The response may be posted anonymously or may identify the individual member.

Additionally, the individual member's reflections can be used to provide the individual member's experience as a social aspect. As a social process, value can be placed on those members of a community that have relevant experience. The reflections from the members can be induced in order to help elevate the aspects of the experience that may be more instructional to the community as a whole. This can help increase the number of members of the community thinking about an action, for example, creating an atmosphere where people can learn about others in who have undergone a change they are considering.

In some embodiments, the method may not include the Block 220.

In embodiments where the response is received and after the response from the individual member is received, the method may repeat the Blocks 216 and 218 to generate a subsequent or second content and/or questions and to transmit a second communication including at least the second content and/or questions to the individual member. The Block 220 may also repeat and a second response may be received from the individual member.

The method described in FIG. 2 can include more or less steps. One or more steps or any combination of steps may also be repeated in the method described in FIG. 2.

One or more embodiments may be implemented on a computing system specifically designed to achieve an improved technological result. When implemented in a computing system, the features and elements of the disclosure provide a significant technological advancement over computing systems that do not implement the features and elements of the disclosure. Any combination of mobile, desktop, server, router, switch, embedded device, or other types of hardware may be improved by including the features and elements described in the disclosure.

For example, as shown in FIG. 3A, the computing system (300) may include one or more computer processor(s) (302), non-persistent storage device(s) (304), persistent storage device(s) (306), a communication interface (308) (e.g., Bluetooth interface, infrared interface, network interface, optical interface, etc.), and numerous other elements and functionalities that implement the features and elements of the disclosure. The computer processor(s) (302) may be an integrated circuit for processing instructions. The computer processor(s) (302) may be one or more cores, or micro-cores, of a processor. The computer processor(s) (302) includes one or more processors. The computer processor(s) (302) may include a central processing unit (CPU), a graphics processing unit (GPU), a tensor processing unit (TPU), combinations thereof, etc.

The input device(s) (310) may include a touchscreen, keyboard, mouse, microphone, touchpad, electronic pen, or any other type of input device. The input device(s) (310) may receive inputs from a user that are responsive to data and messages presented by the output device(s) (312). The inputs may include text input, audio input, video input, etc., which may be processed and transmitted by the computing system (300) in accordance with one or more embodiments. The communication interface (308) may include an integrated circuit for connecting the computing system (300) to a network (not shown) (e.g., a local area network (LAN), a wide area network (WAN) such as the Internet, mobile network, or any other type of network) or to another device, such as another computing device, and combinations thereof.

Further, the output device(s) (312) may include a display device, a printer, external storage, or any other output device. One or more of the output device(s) (312) may be the same or different from the input device(s) (310). The input device(s) (310) and output device(s) (312) may be locally or remotely connected to the computer processor(s) (302). Many different types of computing systems exist, and the aforementioned input device(s) (310) and output device(s) (312) may take other forms. The output device(s) (312) may display data and messages that are transmitted and received by the computing system (300). The data and messages may include text, audio, video, etc., and include the data and messages described above in the other figures of the disclosure.

Software instructions in the form of computer readable program code to perform embodiments may be stored, in whole or in part, temporarily or permanently, on a non-transitory computer readable medium such as a solid-state drive (SSD), compact disk (CD), digital video disk (DVD), storage device, a diskette, a tape, flash memory, physical memory, or any other computer readable storage medium. Specifically, the software instructions may correspond to computer readable program code that, when executed by the computer processor(s) (302), is configured to perform one or more embodiments, which may include transmitting, receiving, presenting, and displaying data and messages described in the other figures of the disclosure.

The computing system (300) in FIG. 3A may be connected to, or be a part of, a network. For example, as shown in FIG. 3B, the network (320) may include multiple nodes (e.g., node X (322) and node Y (324), as well as extant intervening nodes between node X (322) and node Y (324)). Each node may correspond to a computing system, such as the computing system shown in FIG. 3A, or a group of nodes combined may correspond to the computing system shown in FIG. 3A. By way of an example, embodiments may be implemented on a node of a distributed system that is connected to other nodes. By way of another example, embodiments may be implemented on a distributed computing system having multiple nodes, where each portion may be located on a different node within the distributed computing system. Further, one or more elements of the aforementioned computing system (300) may be located at a remote location and connected to the other elements over a network.

The nodes (e.g., node X (322) and node Y (324)) in the network (320) may be configured to provide services for a client device (326). The services may include receiving requests and transmitting responses to the client device (326). For example, the nodes may be part of a cloud computing system. The client device (326) may be a computing system, such as the computing system shown in FIG. 3A. Further, the client device (326) may include or perform all or a portion of one or more embodiments.

The computing system of FIG. 3A may include functionality to present data (including raw data, processed data, and combinations thereof) such as results of comparisons and other processing. For example, presenting data may be accomplished through various presenting methods. Specifically, data may be presented by being displayed in a user interface, transmitted to a different computing system, and stored. The user interface may include a graphical user interface (GUI) that displays information on a display device. The GUI may include various GUI widgets that organize what data is shown, as well as how data is presented to a user. Furthermore, the GUI may present data directly to the user, e.g., data presented as actual data values through text, or rendered by the computing device into a visual representation of the data, such as through visualizing a data model.

Various operations described are purely exemplary and imply no particular order. Further, the operations can be used in any sequence when appropriate and can be partially used. With the above embodiments in mind, it should be understood that additional embodiments can employ various computer-implemented operations involving data transferred or stored in computer systems. These operations are those requiring physical manipulation of physical quantities. Usually, though not necessarily, these quantities take the form of electrical, magnetic, or optical signals capable of being stored, transferred, combined, compared, and otherwise manipulated.

Any of the operations described that form part of the presently disclosed embodiments may be useful machine operations. Various embodiments also relate to a device or an apparatus for performing these operations. The apparatus can be specially constructed for the required purpose, or the apparatus can be a general-purpose computer selectively activated or configured by a computer program stored in the computer. In particular, various general-purpose machines employing one or more processors coupled to one or more computer readable medium, described below, can be used with computer programs written in accordance with the teachings herein, or it may be more convenient to construct a more specialized apparatus to perform the required operations.

The procedures, processes, and/or modules described herein may be implemented in hardware, software, embodied as a computer-readable medium having program instructions, firmware, or a combination thereof. For example, the functions described herein may be performed by a processor executing program instructions out of a memory or other storage device.

The foregoing description has been directed to particular embodiments. However, other variations and modifications may be made to the described embodiments, with the attainment of some or all of their advantages. Modifications to the above-described systems and methods may be made without departing from the concepts disclosed herein. Accordingly, the invention should not be viewed as limited by the disclosed embodiments. Furthermore, various features of the described embodiments may be used without the corresponding use of other features. Thus, this description should be read as merely illustrative of various principles, and not in limitation of the invention.

The present invention may be further exemplified by one, or a combination of one or more of, the following statements:

Statement 1. A method to slow or reverse progression of an adverse health condition, the method comprising: providing a digital communication network (DCN) for members of a population to communicate with each other; sending an offer to an individual member of the population offering an opportunity to reflect on a specific topic related to the adverse health condition; in response to receiving an acceptance of the offer, selecting a piece of music or art to cause the individual member to link an action by the individual member with an outcome the individual member experienced; and sending, through communications on the DCN, a topic message related to the specific topic to the individual member, the topic message including the piece of music or art.

Statement 2. The method of Statement 1, further comprising: receiving a response to the topic message; in response to receiving the response, selecting an additional piece of music or art based on the response to the topic message; and sending, through the communications on the DCN, a subsequent message related to the specific topic to the individual member, the subsequent message including the additional piece of music or art.

Statement 3. The method of Statement 2, wherein selecting the additional piece of music or art is further based on previous conversations between the individual member and the DCN.

Statement 4. The method of Statement 1, wherein the offer and the topic message are sent in a single conversational session.

Statement 5. The method of Statement 1, wherein the offer is sent in a first conversational session and the topic message is sent in a subsequent conversational session.

Statement 6. The method of Statement 5, wherein the first conversational session is held on a first day and the subsequent conversational session is held on a subsequent day.

Statement 7. The method of Statement 1, wherein the topic message is sent by a conversation bot.

Statement 8. The method of Statement 1, further comprising using a conversation bot to generate a question designed to cause the individual member to associate an action with an outcome experienced, wherein the topic message further comprises the question.

Statement 9. The method of Statement 8, wherein the conversation bot is configured to generate textual communications.

Statement 10. The method of Statement 8, wherein the conversation bot is configured to generate verbal communications.

Statement 11. The method of Statement 1, further comprising generating the piece of music or art based, in part, on analysis of communications of the individual member on the DCN.

Statement 12. The method of Statement 11, wherein generating the piece of music or art comprises applying using a large language model (LLM).

Statement 13. The method of Statement 1, wherein the plurality of topics of reflection comprises a biometric reading, a psychological or emotional state, an inspirational quotation identified as useful to the individual member, music, movies, art, prose, or poetry identified as appreciated by the individual member, a daily routine, a life event, a time of year, a season, a time of day, a diagnosis, an injury, an elective medical procedure, a current challenge which the individual member is experiencing, a current challenge which the individual member has taken on, a relationship, a planned trip, and an unplanned trip.

Statement 14. The method of Statement 1, further comprising sending a list of possible topics to the individual member; and receiving a selected topic for the list of possible topics, wherein the selected topic is used as the specific topic used for reflection.

Statement 15. The method of Statement 14, further comprising generating the list of possible topics based in part, through analysis of communications by the individual member on the DCN.

Statement 16. The method of Statement 14, further comprising generating the list of possible topics based, in part, on a phenotype of the individual member.

Statement 17. The method of Statement 16, further comprising determining the phenotype of the individual member through analysis of communications by the individual member on the DCN.

Statement 18. The method of Statement 1, wherein the topic message identifies an incentive for the individual member to reflect on the specific topic.

Statement 19. The method of Statement 18, wherein the incentive is non-monetary.

Statement 20. The method of Statement 1, further comprising receiving a reflection response to the topic message and posting the reflection response to the DCN.

Statement 21. The method of Statement 1, further comprising generating the topic message based, at least in part, on prior communications by the individual member on the DCN.

Statement 22. The method of Statement 1, further comprising analyzing communications in the DCN to determine the adverse health condition.

Statement 23. The method of Statement 1, further comprising analyzing communication in the DCN to generate an ontology of health conditions for the individual member, wherein the ontology of health conditions comprises the adverse health condition.

Statement 24. The method of Statement 23, wherein analyzing communications in the DCN comprises applying a large language model to the communications in the DCN.

Statement 25. The method of Statement 1, wherein the adverse health condition comprises at least one of: chronic systemic inflammation, malaise, and low energy.

Statement 26. The method of Statement 1, wherein the adverse health condition comprises a disease.

Statement 27. The method of Statement 26, wherein the disease comprises at least one of: obesity, diabetes, rheumatoid arthritis (RA), Crohn's disease, psoriasis, eczema, cardiovascular disease (CVD), congestive heart failure (CHF), chronic obstructive pulmonary disease (COPD), insomnia, sleep quality disorders, asthma, depression, and anxiety.

Statement 28. The method of Statement 1, wherein the adverse health condition comprises a prodromal version of a disease.

Statement 29. The method of Statement 1, wherein the adverse health condition comprises a health risk.

Statement 30. The method of Statement 29, wherein the health risk comprises at least one of: a risk of falling, and a risk of becoming infected.

Statement 31. The method of Statement 1, wherein the adverse health condition comprises social dysfunction.

Statement 32. The method of Statement 1, wherein communications in the DCN comprise at least one of: one-to-one communications, one-to-many communications, one-to-system communications, system-to-one communications, and system-to-many communications.

Statement 33. A method to improve communications with a conversation bot, the method comprising: providing a digital communication network (DCN) for members of a population to communicate with each other; sending an offer to an individual member of the population offering an opportunity to reflect on a specific topic related to an adverse health condition; in response to receiving acceptance of the offer, using an artificial intelligence (AI)-driven bot to generate a piece of artistic expression designed to cause the individual member to link an action by the individual member with an outcome the individual member experienced; and sending, through communications on the DCN, a topic message related to the specific topic to the individual member, the topic message including the piece of artistic expression.

Statement 34. The method of Statement 33, wherein the piece of artistic expression comprises a lyrical piece of music.

Statement 35. The method of Statement 34, wherein the lyrical piece of music comprises a line of an opera.

Statement 36. The method of Statement 33, wherein the piece of artistic expression comprises a choreographed dance, the topic message including a video composition showing the choreographed dance.

Statement 37. The method of Statement 33, further comprising: receiving a response to the topic message; in response to receiving the response, using the AI-driven bot to generate an additional piece of artistic expression based on the response to the topic message; and sending, through the communications on the DCN, a subsequent message related to the specific topic to the individual member, the subsequent message including the additional piece of artistic expression.

Statement 38. The method of Statement 37, wherein the AI-driven bot generates the additional piece of artistic expression based on previous conversations between the individual member and the DCN.

Statement 39. The method of Statement 33, wherein the offer and the topic message are sent in a single conversational session.

Statement 40. The method of Statement 33, wherein the offer is sent in a first conversational session and the topic message is sent in a subsequent conversational session.

Statement 41. The method of Statement 40, wherein the first conversational session is held on a first day and the subsequent conversational session is held on a subsequent day.

Statement 42. The method of Statement 33, wherein the topic message is sent by a conversation bot.

Statement 43. The method of Statement 33, further comprising using a conversation bot to generate a question designed to cause the individual member to associate an action with an outcome experienced, wherein the topic message further comprises the question.

Statement 44. The method of Statement 43, wherein the conversation bot in configured to generate textual communications.

Statement 45. The method of Statement 43, wherein the conversation bot in configured to generate verbal communications.

Statement 46. The method of Statement 33, wherein the AI-driven bot generates the piece of artistic expression based, in part, on analysis of communications of the individual member on the DCN.

Statement 47. The method of Statement 46, wherein the AI-driven bot generates the piece of artistic expression using a large language model (LLM).

Statement 48. The method of Statement 33, wherein the specific topic is at least one of: a biometric reading, a psychological or emotional state, an inspirational quotation identified as useful to the individual member, music, movies, art, prose, or poetry identified as appreciated by the individual member, a daily routine, a life event, a time of year, a season, a time of day, a diagnosis, an injury, an elective medical procedure, a current challenge which the individual member is experiencing, a current challenge which the individual member has taken on, a relationship, a planned trip, and an unplanned trip.

Statement 49. The method of Statement 33, further comprising sending a list of possible topics to the individual member; and receiving a selected topic for the list of possible topics, wherein the selected topic is used as the specific topic used for reflection.

Statement 50. The method of Statement 49, further comprising generating the list of possible topics based in part, through analysis of communications by the individual member on the DCN.

Statement 51. The method of Statement 49, further comprising generating the list of possible topics based, in part, on a phenotype of the individual member.

Statement 52. The method of Statement 51, further comprising determining the phenotype of the individual member through analysis of communications by the individual member on the DCN.

Statement 53. The method of Statement 33, wherein the topic message identifies an incentive for the individual member to reflect on the specific topic.

Statement 54. The method of Statement 53, wherein the incentive is non-monetary.

Statement 55. The method of Statement 33, further comprising receiving a reflection response to the topic message and posting the reflection response to the DCN.

Statement 56. The method of Statement 33, further comprising generating the topic message based, at least in part, on prior communications by the individual member on the DCN.

Statement 57. The method of Statement 33, further comprising analyzing communications in the DCN to determine the adverse health condition.

Statement 58. The method of Statement 33, further comprising analyzing communication in the DCN to generate an ontology of health conditions for the individual member, wherein the ontology of health conditions comprises the adverse health condition.

Statement 59. The method of Statement 58, wherein analyzing communications in the DCN comprises applying a large language model to the communications in the DCN.

Statement 60. The method of Statement 33, wherein the adverse health condition comprises at least one of: chronic systemic inflammation, malaise, and low energy.

Statement 61. The method of Statement 33, wherein the adverse health condition comprises a disease.

Statement 62. The method of Statement 61, wherein the disease comprises at least one of: obesity, diabetes, rheumatoid arthritis (RA), Crohn's disease, psoriasis, eczema, cardiovascular disease (CVD), congestive heart failure (CHF), chronic obstructive pulmonary disease (COPD), insomnia, sleep quality disorders, asthma, depression, and anxiety.

Statement 63. The method of Statement 33, wherein the adverse health condition comprises a prodromal version of a disease.

Statement 64. The method of Statement 33, wherein the adverse health condition comprises a health risk.

Statement 65. The method of Statement 64, wherein the health risk comprises at least one of: a risk of falling, and a risk of becoming infected.

Statement 66. The method of Statement 33, wherein the adverse health condition comprises social dysfunction.

Statement 67. The method of Statement 33, wherein communications in the DCN comprise at least one of: one-to-one communications, one-to-many communications, one-to-system communications, system-to-one communications, and system-to-many communications.

Statement 68. A method to improve communications with a conversation bot, the method comprising: providing a digital communication network (DCN) for members of a population to communicate with each other; in response to a communication from an individual member of the population, using an artificial intelligence (AI)-driven bot to generate a piece of artistic expression designed to cause the individual member to link an action by the individual member with an outcome the individual member experienced; and sending, through communications on the DCN, an artistic message, the artistic message including the piece of artistic expression.

Statement 69. The method of Statement 68, wherein the piece of artistic expression comprises a lyrical piece of music.

Statement 70. The method of Statement 69, wherein the lyrical piece of music comprises a line of an opera.

Statement 71. The method of Statement 68, wherein the piece of artistic expression comprises a choreographed dance, the artistic message including a video composition showing the choreographed dance.

Statement 72. The method of Statement 68, further comprising: receiving a response to the artistic message; in response to receiving the response, using the AI-driven bot to generate an additional piece of artistic expression based on the response to the artistic message; and sending, through the communications on the DCN, a subsequent message to the individual member, the subsequent message including the additional piece of artistic expression.

Statement 73. The method of Statement 72, wherein the AI-driven bot generates the additional piece of artistic expression based on previous conversations between the individual member and the DCN.

Statement 74. The method of Statement 68, wherein the communication from the individual member and the artistic message are sent in a single conversational session.

Statement 75. The method of Statement 68, wherein the communication from the individual member is sent in a first conversational session and the artistic message is sent in a subsequent conversational session.

Statement 76. The method of Statement 75, wherein the first conversational session is held on a first day and the subsequent conversational session is held on a subsequent day.

Statement 77. The method of Statement 68, wherein the artistic message is sent by a conversation bot.

Statement 78. The method of Statement 68, further comprising using a conversation bot to generate a question designed to cause the individual member to associate an action with an outcome experienced, wherein the artistic message further comprises the question.

Statement 79. The method of Statement 78, wherein the conversation bot in configured to generate textual communications.

Statement 80. The method of Statement 78, wherein the conversation bot in configured to generate verbal communications.

Statement 81. The method of Statement 68, wherein the AI-driven bot generates the piece of artistic expression based, in part, on analysis of communications of the individual member on the DCN.

Statement 82. The method of Statement 81, wherein the AI-driven bot generates the piece of artistic expression using a large language model (LLM).

Statement 83. The method of Statement 68, wherein the artistic message identifies an incentive for the individual member to reflect.

Statement 84. The method of Statement 83, wherein the incentive is non-monetary.

Statement 85. The method of Statement 68, further comprising receiving a reflection response to the artistic message and posting the reflection response to the DCN.

Statement 86. The method of Statement 68, further comprising generating the artistic message based, at least in part, on prior communications by the individual member on the DCN.

Statement 87. The method of Statement 68, further comprising analyzing communications in the DCN to determine an adverse health condition of the individual member.

Statement 88. The method of Statement 87, further comprising analyzing communication in the DCN to generate an ontology of health conditions for the individual member, wherein the ontology of health conditions comprises the adverse health condition.

Statement 89. The method of Statement 88, wherein analyzing communications in the DCN comprises applying a large language model to the communications in the DCN.

Statement 90. The method of Statement 87, wherein the adverse health condition comprises at least one of: chronic systemic inflammation, malaise, and low energy.

Statement 91. The method of Statement 87, wherein the adverse health condition comprises a disease.

Statement 92. The method of Statement 91, wherein the disease comprises at least one of: obesity, diabetes, rheumatoid arthritis (RA), Crohn's disease, psoriasis, eczema, cardiovascular disease (CVD), congestive heart failure (CHF), chronic obstructive pulmonary disease (COPD), insomnia, sleep quality disorders, asthma, depression, and anxiety.

Statement 93. The method of Statement 87, wherein the adverse health condition comprises a prodromal version of a disease.

Statement 94. The method of Statement 87, wherein the adverse health condition comprises a health risk.

Statement 95. The method of Statement 94, wherein the health risk comprises at least one of: a risk of falling, and a risk of becoming infected.

Statement 96. The method of Statement 87, wherein the adverse health condition comprises social dysfunction.

Statement 97. The method of Statement 68, wherein communications in the DCN comprise at least one of: one-to-one communications, one-to-many communications, one-to-system communications, system-to-one communications, and system-to-many communications.

Statement 98. A method to improve communications with a conversation bot, the method comprising: providing a digital communication network (DCN) for members of a population to communicate with each other; receiving a prompt from an individual member of the population; in response to receiving the prompt, generating a lyrical response to the prompt; generating an audible message to the prompt, wherein the audible message comprises the lyrical response and a musical component; and sending, through communications on the DCN, the audible message to the individual member.

Statement 99. The method of Statement 98, further comprising selecting the musical component to add an emotional boost that reinforces the lyrical response.

Statement 100. The method of Statement 98, wherein the audible message is designed to at least one of: improve health of the individual member, manage a disease of the individual member, and increase wellbeing of the individual member.

Statement 101. The method of Statement 98, further comprising: receiving a reply to the audible message; in response to receiving the reply, using the AI-driven bot to generate an additional lyrical response based on the reply to the audible message generating a subsequent audible message to the prompt, wherein the subsequent audible message comprises the additional lyrical response and an additional musical component; and sending, through the communications on the DCN, the subsequent audible message to the individual member.

Statement 102. The method of Statement 101, wherein the AI-driven bot generates the additional lyrical response based on previous conversations between the individual member and the DCN.

Statement 103. The method of Statement 98, wherein the prompt and the audible message are sent in a single conversational session.

Statement 104. The method of Statement 98, wherein the prompt is sent in a first conversational session and the audible message is sent in a subsequent conversational session.

Statement 105. The method of Statement 104, wherein the first conversational session is held on a first day and the subsequent conversational session is held on a subsequent day.

Statement 106. The method of Statement 98, wherein the audible message is sent by a conversation bot.

Statement 107. The method of Statement 98, further comprising using a conversation bot to generate a question designed to cause the individual member to associate an action with an outcome experienced, wherein the audible message further comprises the question.

Statement 108. The method of Statement 98, further comprising using a conversation bot to generate a question designed to cause the individual member to associate an action with an outcome experienced, wherein the lyrical response further comprises the question.

Statement 109. The method of Statement 98, wherein the AI-driven bot generates the lyrical response based, in part, on analysis of communications of the individual member on the DCN.

Statement 110. The method of Statement 109, wherein the AI-driven bot generates the lyrical response using a large language model (LLM).

Statement 111. The method of Statement 98, wherein the audible message identifies an incentive for the individual member to reflect.

Statement 112. The method of Statement 111, wherein the incentive is non-monetary.

Statement 113. The method of Statement 98, further comprising receiving a reflection response to the audible message and posting the reflection response to the DCN.

Statement 114. The method of Statement 98, further comprising generating the audible message based, at least in part, on prior communications by the individual member on the DCN.

Statement 115. The method of Statement 98, further comprising analyzing communication in the DCN to generate an ontology of health conditions for the individual member.

Statement 116. The method of Statement 115, wherein analyzing communications in the DCN comprises applying a large language model to the communications in the DCN.

Statement 117. The method of Statement 98, further comprising analyzing communications in the DCN to determine an adverse health condition of the individual member.

Statement 118. The method of Statement 117, wherein the adverse health condition comprises at least one of: chronic systemic inflammation, malaise, and low energy.

Statement 119. The method of Statement 117, wherein the adverse health condition comprises a disease.

Statement 120. The method of Statement 119, wherein the disease comprises at least one of: obesity, diabetes, rheumatoid arthritis (RA), Crohn's disease, psoriasis, eczema, cardiovascular disease (CVD), congestive heart failure (CHF), chronic obstructive pulmonary disease (COPD), insomnia, sleep quality disorders, asthma, depression, and anxiety.

Statement 121. The method of Statement 117, wherein the adverse health condition comprises a prodromal version of a disease.

Statement 122. The method of Statement 117, wherein the adverse health condition comprises a health risk.

Statement 123. The method of Statement 122, wherein the health risk comprises at least one of: a risk of falling, and a risk of becoming infected.

Statement 124. The method of Statement 117, wherein the adverse health condition comprises social dysfunction.

Statement 125. The method of Statement 98, wherein communications in the DCN comprise at least one of: one-to-one communications, one-to-many communications, one-to-system communications, system-to-one communications, and system-to-many communications.

What is claimed is:

1. A method comprising:
providing a digital communication network (DCN) for a plurality of members of a population to communicate with at least each other or the DCN;
identifying, by a server controller, an individual member with an adverse health condition of the plurality of members;
transmitting, by the server controller, a request to the individual member, via the DCN, to select a topic of reflection from a plurality of topics of reflection corresponding to the adverse health condition;
automatically generating, by the server controller, content comprising at least one of audible content, visual content, or an audible and visual content based on the selected topic of reflection, the content designed to cause the individual member to connect an outcome experienced by the individual member with a decision the individual member has made; and
transmitting a communication generated by the server controller to the individual member via the DCN, the communication including the content and the selected topic of reflection.

2. The method of claim 1, wherein the content is a first content and the communication generated by the server controller is a first communication, wherein the method further comprises:
receiving a response from the individual member to the first communication generated by the server controller via the DCN;
generating, by the server controller, a second content in response to receiving the response; and
transmitting a second communication generated by the server controller to the individual member via the DCN, the second communication including the second content.

3. The method of claim 2, wherein the second content is generated based on previous communications between the individual member and a conversation bot of the DCN.

4. The method of claim 1, further comprising:
generating, by the server controller, a question designed to cause the individual member to associate an action with an outcome experienced, wherein the communication generated by the server controller includes the question.

5. The method of claim 1, further comprising:
analyzing, by the server controller, one or more communications from the individual member, wherein the content is automatically generated based in part of the analysis of the one or more communications.

6. The method of claim 1, further comprising:
generating, by the server controller, a prompt containing at least the selected topic of reflection and instructions to generate the topic, wherein automatically generating the content comprises prompting a large language model with the prompt and receiving the content as output from the large language model.

7. The method of claim 1, wherein the plurality of topics of reflection comprises a biometric reading, a psychological or emotional state, an inspirational quotation identified as useful to the individual member, music, movies, art, prose, or poetry identified as appreciated by the individual member, a daily routine, a life event, a time of year, a season, a time of day, a diagnosis, an injury, an elective medical procedure, a current challenge which the individual member is experiencing, a current challenge which the individual member has taken on, a relationship, a planned trip, and an unplanned trip.

8. The method of claim 1, further comprising:
analyzing, by the server controller, one or more communications from the individual member; and
generating, by the server controller, the plurality of topics of reflection based on the analysis of the one or more communications.

9. The method of claim 1, further comprising:
analyzing, by the server controller, one or more communications from the individual member; and
determining, by the server controller, the adverse health condition based on the analysis of the one or more communications.

10. The method of claim 1, wherein the content comprises at least one of a piece of music, a piece of art, a lyrical piece of music, a line of a song, a line of an opera, a video showing a choreographed dance, a song, a painting, a mural, mixed media content, and a combination of music with a piece of art.

11. A system comprising:
a computer processor;
a data repository in communication with the computer processor and storing:
a request;
a plurality of topics of reflection;
a content; and
a communication;
a digital communications network (DCN) which, when executed by the computer processor, provides a network for a plurality of members of a population to interact with at least each other or the DCN; and
a server controller which, when executed by the computer processor:
identifies an individual member with an adverse health condition from the plurality of members;
transmits the request to the individual member, via the DCN, to select a topic of reflection from the plurality of topics of reflection corresponding to the adverse health condition;
automatically generates content comprising at least one of audible content, visual content, or an audible and visual content based on the selected topic of reflection, the content designed to cause the individual member to connect an outcome experienced by the individual member with a decision the individual member has made; and
transmits the communication to the individual member via the DCN, the communication including the content and the selected topic of reflection.

12. The system of claim 11, wherein the content is a first content and the communication is a first communication, and wherein the server controller when further executed by the processor:
receives a response from the individual member to the first communication via the DCN;
generates a second content in response to receiving the response; and
transmits a second communication to the individual member via the DCN, the second communication including the second content.

13. The system of claim 12, wherein the second content is generated based on previous communications between the individual member and a conversation bot of the DCN.

14. The system of claim 11, wherein the server controller when further executed by the processor:
generates a question designed to cause the individual member to associate an action with an outcome experienced, wherein the communication generated by the server controller includes the question.

15. The system of claim 11, wherein the server controller when further executed by the processor:
analyzes one or more communications from the individual member, wherein the content is automatically generated based in part of the analysis of the one or more communications.

16. The system of claim 11, wherein the server controller when further executed by the processor:
generates a prompt containing at least the selected topic of reflection and instructions to generate the topic, wherein automatically generating the content comprises prompting a large language model with the prompt and receiving the content as output from the large language model.

17. The system of claim 11, wherein the plurality of topics of reflection comprises a biometric reading, a psychological or emotional state, an inspirational quotation identified as useful to the individual member, music, movies, art, prose, or poetry identified as appreciated by the individual member, a daily routine, a life event, a time of year, a season, a time of day, a diagnosis, an injury, an elective medical procedure, a current challenge which the individual member is experiencing, a current challenge which the individual member has taken on, a relationship, a planned trip, and an unplanned trip.

18. The system of claim 11, wherein the server controller when further executed by the processor:
analyzes one or more communications from the individual member; and
generates the plurality of topics of reflection based on the analysis of the one or more communications.

19. The system of claim 11, wherein the server controller when further executed by the processor:
analyzes one or more communications from the individual member; and
determines the adverse health condition based on the analysis of the one or more communications.

20. The system of claim 11, wherein the content comprises at least one of a piece of music, a piece of art, a lyrical piece of music, a line of a song, a line of an opera, a video showing a choreographed dance, a song, a painting, a mural, mixed media content, and a combination of music with a piece of art.
